(19) United States
(12) Patent Application Publication
(10) Pub. No.: US 2023/0248373 A1
(43) Pub. Date: Aug. 10, 2023

(54) SURGICAL AIMING DEVICE

(71) Applicant: JEIL MEDICAL CORPORATION, Seoul (KR)

(72) Inventors: Geung-Rok KIM, Seoul (KR); Chi-Hoon KIM, Goyang-si, Gyeonggi-do (KR); Jang-Hyeon SEO, Gimpo-si, Gyeonggi-do (KR)

(21) Appl. No.: 18/004,319

(22) PCT Filed: Feb. 5, 2021
(86) PCT No.: PCT/KR2021/001575

§ 371 Date: Jan. 5, 2023

(30) Foreign Application Priority Data
Jul. 9, 2020 (KR) 10-2020-0084625

(51) Int. Cl. A61B 17/17

(52) U.S. Cl. CPC A61B 17/1717; A61B 2017/00367

(57) ABSTRACT

The present disclosure relates to a surgical aiming device 1 that prevents a sleeve from being pushed backward or shaken when a drill or a leg screw is implanted after a guide sleeve is mounted on an aiming device. The surgical aiming device includes: a body 20 having an aiming arm 21 and a barrel 22; a guide sleeve 30 disposed at an end of the aiming arm 21 and guiding a nail 10 and a leg screw 12; and a knob 40 for detachably attaching the guide sleeve 30, in which the guide sleeve 30 can be detachably attached in a one-touch type by a guide sleeve attachment/detachment unit 100 installed at an end of the aiming arm. The guide sleeve attachment/detachment unit 100 includes: an operation unit 21a installed at an end of the aiming arm 21; a button 110 installed at the operation unit 21a and partially exposed to the outside; an operation lever 120 being rotated on a rotary shaft by operation of the button 110; and a threaded portion 30a controlled by the operation lever 120 and formed on the guide sleeve 30a.

### TECHNICAL FIELD

The present disclosure relates to a surgical aiming device, and more specifically, to a surgical aiming device that prevents a sleeve from being pushed backward or shaken when a drill or a leg screw is implanted after a guide sleeve is mounted on an aiming device.

### BACKGROUND ART

Fixation and stabilization of a bone in a creature is generally associated with an implant (e.g., an intramedullary nail) that is inserted in a target bone. Mechanical aiming tools are sometimes used to help align an implant on or in a target bone.

Such aiming devices generally have a protection sleeve that is inserted through a hole formed toward an implant to provide a barrier for maintaining a necessary position of an implant with respect to the aiming device and preventing an injury of a soft tissues during implanting. Such a protection sleeve generally has an opening to introduce a drill, a screw, and other tools, or implants therethrough.

At present, available aiming devices provide a tension mechanism such as a spring, a set-screw, and a spring-load contact element configured to tense the outer wall of a protection sleeve when they are inserted through an aiming arm.

In particular, such a tension mechanism should be able to apply predetermined friction to the protection sleeve against an implant or should be able to be selectively coupled or decoupled by an alternative during the entire process in which it is inserted through an aiming tool.

That is, an aiming device 1, as shown in FIG. 1, is a device serving to target a leg screw 12 coupled to a nail 10, in which an aiming arm 21 is disposed on a side of a body and a barrel 22 is disposed on another side, a guide sleeve 30 that guides the nail 10 and the leg screw 12 is disposed at an end of the aiming arm 21, and the guide sleeve 30 is detachably attached by a knob 40.

However, the aiming arm 21 generally prevent shaking or pushing by tightening the knob 40 when the guide sleeve 30 is fastened, but there is a trouble that it is necessarily required to tighten the knob 40 in order to fix the guide sleeve 30 and loosen the knob 40 in order to detach the guide sleeve 30.

### DISCLOSURE

### Technical Problem

Accordingly, the present disclosure has been made in an effort to solve the general problems of the related art described above and an objective is to provide a surgical aiming device that can prevent a sleeve from being pushed backward or shaken when a drill or a leg screw is implanted after a guide sleeve is mounted on a surgical aiming device, and can be easily detached through a button touch.

### Technical Solution

In order to achieve the objectives of the present disclosure,

a surgical aiming device for aiming a leg screw to aim a leg screw coupled to a nail includes:

a body having an aiming arm and a barrel;

a guide sleeve disposed at an end of the aiming arm and guiding a nail and a leg screw; and

a knob for detachably attaching the guide sleeve, wherein the guide sleeve can be detachably attached in a one touch type by a guide sleeve attachment/detachment unit installed at an end of the aiming arm.

The guide sleeve attachment/detachment unit includes:

an operation unit installed at an end of the aiming arm;

a button installed at the operation unit and partially exposed to the outside;

an operation lever being rotated on a rotary shaft by operation of the button; and

a threaded portion controlled by the operation lever and formed on the guide sleeve.

In addition, the operation lever has an elastic member seat at an end in which an elastic member that is contracted and restored by the operation level when the button is pressed, and has a locking portion formed at an end corresponding to the elastic member.

### Advantageous Effects

The surgical aiming device of the present disclosure having the configuration described above is composed of a thread, a thread locking portion, and an elastic body and applies shaking, anti-pushing, rotation, and attachment/detachment, whereby it can prevent pushing by only pushing a guide sleeve inside and it can be easily attached/detached by only a button touch.

### DESCRIPTION OF DRAWINGS

FIG. 1 is a view showing a surgical aiming device of the related art.

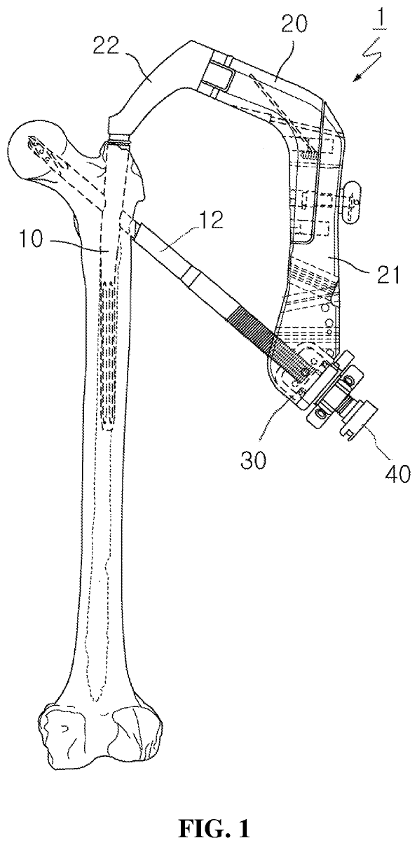

FIG. 2 is a view showing a surgical aiming device according to the present disclosure.

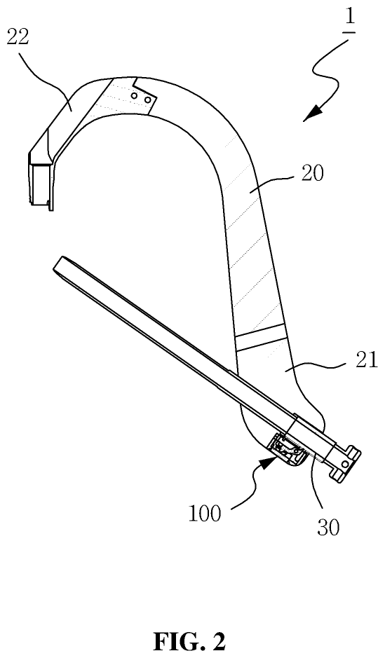

FIG. 3 is a view showing only a guide sleeve attachment/detachment unit of the surgical aiming device according to the present disclosure.

### BEST MODE

Hereinafter, the configuration of a surgical aiming device according to the present disclosure will be described in detail with reference to the accompanying drawings.

However, the disclosed drawings are provided as examples so that the spirit of the present disclosure can be sufficiently transmitted to those skilled in the art. Accordingly, the present disclosure is not limited to the proposed drawings and may be implemented by other ways.

Further, unless stated otherwise, the terms used herein have meanings that those skilled in the art generally understand, and well-known functions and configurations that may make the main idea of the present disclosure unclear in the following description and the drawings are not described in detail.

The present disclosure is a surgical aiming device 1 for aiming a leg screw 12 coupled to a nail 10, which is composed of: a body 20 having an aiming arm 21 and a barrel 22; a guide sleeve 30 disposed at an end of the aiming arm 21 and guiding the nail 10 and the leg screw 12, and a knob 40 for detachably attaching the guide sleeve 30, which is the same as the configuration of an aiming device of the related art.

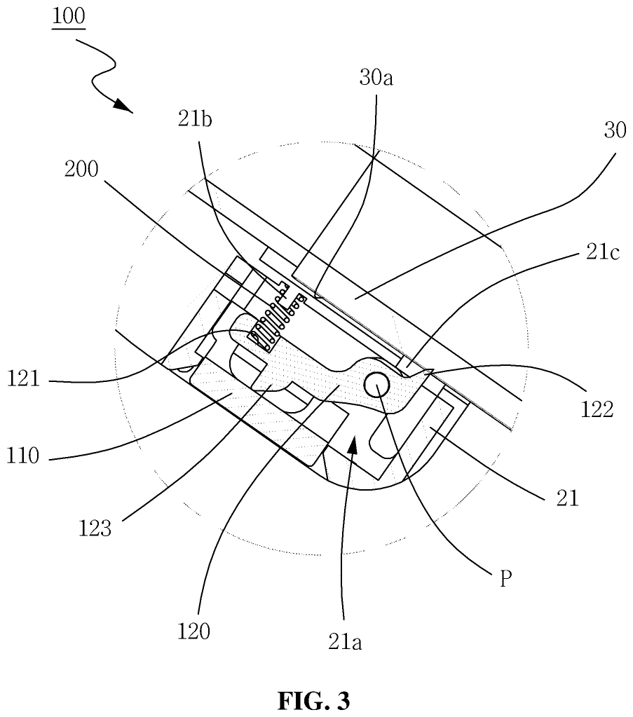

However, as shown in FIG. 3, the present disclosure is characterized in that the guide sleeve 30 can be detachably attached in a one touch type by a guide sleeve attachment/detachment unit 100 installed at an end of the aiming arm 21.

In more detail, the guide sleeve attachment/detachment unit 100 has an operation unit 21a installed at an end of the aiming arm 21.

The operation unit 21a includes a button 110, an operation lever 120, and an elastic member 200 to be described below, and an accommodation space for accommodating them.

The button 110 is installed to be partially exposed out of the operation unit 21a, and is designed to be partially in contact with the operation lever 120.

The operation lever 120 is rotatably fitted on a rotary shaft P formed in the operation unit 210a, in which an elastic member seat 121 for installing an elastic member 200 is formed at an end thereof, a protruding locking portion 122 is formed at another end, and a pressing protrusion 123 for easily pressing the button 110 is disposed on the top of the elastic member seat 121.

The locking portion 122 is formed in a shape with a pointed front end and is locked to a threaded portion30a of the guide sleeve 30.

Further, the elastic member 200 inserted and installed in the elastic member seat 121 formed in the operation lever 120 is a compression spring, and the elastic member 200 is installed and fixed to a spring fixing portion 21b formed in the seat of the operation unit 21a.

Meanwhile, the locking portion 122 protruding from the operation lever 120 is configured to be locked to the threaded portion 30a formed at the guide sleeve 30 while moving inside and outside a hole 21c formed in the operation unit 21a.

Accordingly, it is possible to control attachment/detachment of the guide sleeve in a one touch type using the guide sleeve attachment/detachment unit 100 having the configuration described above.

In more detail, the guide sleeve 30 is thread-fastened to a threaded portion (not shown) formed on the aiming arm 21, and in this process, the guide sleeve 30 slides over the locking portion 122 formed at an end of the operation lever 120.

In this state, when the button 110 is pressed, the operation lever 120 presses the elastic member 200, rotates clockwise about the rotary shaft P, and separates from the threaded portion 30a formed on the guide sleeve 30, whereby the guide sleeve 30 can be easily separated.

Although the configuration and rotation of a surgical aiming device of the present disclosure were described above, the present disclosure may be changed, modified, and replaced in various ways by those skilled in the art, and the change, modification, and replacement should be construed as being included in the protective range of the present disclosure.

[Description of the Reference Numerals]

| | |
|---|---|
| 21: aiming arm | 21a: operation unit |
| 30: guide sleeve | 30a: threaded portion |
| 100: guide sleeve attachment/detachment unit | |
| 110: button | 120: operation lever |
| 200: elastic member | |

1. A surgical aiming device for aiming a leg screw coupled to a nail, comprising:
a body having an aiming arm and a barrel;
a guide sleeve disposed at an end of the aiming arm and guiding a nail and a leg screw; and
a knob for detachably attaching the guide sleeve,
wherein the guide sleeve can be detachably attached in a one-touch type by a guide sleeve attachment/detachment unit installed at an end of the aiming arm.

2. The surgical aiming device of claim 1, wherein the guide sleeve attachment/detachment unit comprises:
an operation unit installed at an end of the aiming arm;
a button installed at the operation unit and partially exposed to the outside;
an operation lever being rotated on a rotary shaft by operation of the button; and
a threaded portion controlled by the operation lever and formed on the guide sleeve.

3. The surgical aiming device of claim 2, wherein the operation lever has an elastic member seat at an end in which an elastic member that is contracted and restored by the operation level when the button is pressed, and has a locking portion formed at an end corresponding to the elastic member.
